(19) United States
(12) Patent Application Publication
(10) Pub. No.: US 2024/0053334 A1
(43) Pub. Date: Feb. 15, 2024

(54) SYSTEM AND METHOD FOR GRAVITY COMPENSATION IN A SENSOR SYSTEM

(71) Applicant: Siemens Healthineers Nederland B.V., Den Haag (NL)

(72) Inventors: Alexander van Reenen, Vught (NL); Jeroen Hans Nieuwenhuis, Boston, MA (US)

(73) Assignee: Siemens Healthineers Nederland B.V., Den Haag (NL)

(21) Appl. No.: 18/258,195

(22) PCT Filed: Dec. 9, 2021
(86) PCT No.: PCT/EP2021/084964

§ 371 Date: Jun. 16, 2023

Related U.S. Application Data
(60) Provisional application No. 63/127,384, filed on Dec. 18, 2020.

(51) Int. Cl. G01N 33/543; G01N 21/552; G01N 21/01

(52) U.S. Cl. CPC G01N 33/54333; G01N 21/552; G01N 21/01; G01N 33/54373

(57) ABSTRACT

Methods and systems are provided for compensating for gravitational effects on a sensor system. In one example, a sensor system includes a sample container configured to receive a sample containing an analyte to be tested, the sample container comprising a detection surface and a plurality of signal generating elements in the sample container, wherein the detection surface comprises a binding surface, which has been partially functionalized with capture elements that can bind, directly and/or indirectly, the analyte and/or the signal generating elements, wherein the signal generating elements have a spatial distribution profile over the detection surface, wherein the spatial distribution profile has a gradient along a first axis, and wherein the binding surface has an axis of symmetry that is orthogonal to the first axis.

### FIELD OF TECHNOLOGY

The present description relates generally to systems and methods for a sensor system for the detection of target molecules in a sample, and more specifically to compensating for gravitational effects on the detection of target molecules.

### BACKGROUND

Biosensors may allow for the detection of a given specific molecule within a sample, wherein the amount or concentration of said specific molecule is typically small, sometimes in the range of nanograms per milliliter. To detect these molecules, functionalized labels or detection tags, such as enzymes, fluorophores, or magnetic beads, may be utilized. In a magnetic-label biosensor, measuring the presence of certain biochemical agents such as drugs or cardiac markers is based on molecular capture and labeling with magnetic particles or beads. Magnetic attraction of the beads, also referred to as actuation, may increase the performance, e.g., speed, of the biosensor for point-of-care applications. The direction of the magnetic attraction can be either towards the surface where the actual measurement is carried out or away from this surface. In the first case magnetic actuation allows the enhancement of the concentration of magnetic particles near the sensor surface (where the magnetic particles may bind to a corresponding capture element, such as an antibody, on the sensor surface), speeding up the binding process of the magnetic particles at the sensor surface. In the second case, unbound magnetic particles (e.g., magnetic particles that are not bound to a capture element on the sensor surface) are removed from the surface which is called magnetic washing.

### SUMMARY

In one embodiment, a sensor system includes a sample container configured to receive a sample containing an analyte to be tested, the sample container comprising a detection surface and a plurality of signal generating elements in the sample container, wherein the detection surface comprises a binding surface, which has been partially functionalized with capture elements that can bind, directly and/or indirectly, the analyte and/or the signal generating elements, wherein the signal generating elements have a spatial distribution profile over the detection surface, wherein the spatial distribution profile has a gradient along a first axis, and wherein the binding surface has an axis of symmetry that is orthogonal to the first axis.

To the accomplishment of the foregoing and related ends, certain illustrative aspects of the system are described herein in connection with the following description and the attached drawings. The features, functions, and advantages that have been discussed can be achieved independently in various embodiments of the present disclosure or may be combined in yet other embodiments, further details of which can be seen with reference to the following description and drawings. The summary is provided to introduce a selection of concepts in a simplified form that are further described below in the Detailed Description. This summary is not intended to identify key features or essential features of any subject matter described herein.

### BRIEF DESCRIPTION OF THE DRAWINGS

FIG. 1 schematically shows a general setup of a sensor system according to the present disclosure.

FIGS. 2-5 schematically show an example sample cartridge of a sensor system having one magnetic element according to the present disclosure.

FIGS. 6-8B schematically show example signal generating element distributions within the sample cartridge of FIGS. 2-5 according to the present disclosure.

FIGS. 9 and 10 schematically show example binding surface layouts within sample cartridges of a sensor system according to the present disclosure.

FIGS. 11-12B schematically show example signal generating element distributions within a sample cartridge of a sensor system having two magnetic elements according to the present disclosure.

FIG. 13 is a flow chart illustrating a method for testing a sample with a sensor system according to the present disclosure.

FIG. 14 schematically shows a sensor system in use in a moving vehicle according to the present disclosure.

### DETAILED DESCRIPTION

The following description relates to systems and methods for a sensor system, also referred to as a microfluidic testing system or microelectronic sensor system. The sensor system may be a magnetic sensor system including a sample container having one or more reaction chambers loaded with functionalized magnetic particles, e.g., antibody-labeled magnetic particles, that are configured to bind to a specific target molecule (also referred to herein as an analyte or analyte of interest) such as troponin or B-type natriuretic peptide (BNP). In some examples, the sample container may be loaded with two or more sets of functionalized magnetic particles, where each set is functionalized for a specific analyte. For example, a first set of magnetic particles may be functionalized with an antibody specific to troponin and a second set of magnetic particles may be functionalized with an antibody specific to BNP. Each reaction chamber has a detection surface that is also functionalized, e.g., with the same and/or different antibodies as those bound to the magnetic particles, thereby forming a binding surface on the detection surface. In this way, the magnetic particles may bind to the binding surface via the specific analyte(s), with the number of magnetic particles that bind to the binding surface being a function of the concentration of each analyte.

One or more magnetic elements may be positioned outside the sample container (e.g., below the sample container), and a magnetic field generated by the one or more magnetic elements may attract the magnetic particles to the binding surface to expedite binding of the magnetic particle/analyte complexes to the binding surface. The area of the binding surface that binds the magnetic particle/analyte complexes may thus be based on the size and position of the magnetic element, as well any variations in the magnetic field generated by the magnetic element. Typically, the antibodies/capture elements are fixed to the detection surface in discrete regions, such as discrete patches or spots. Further, some sample containers may be configured to detect the concentration of more than one analyte, and thus different capture elements may be present in different binding surface regions.

Accordingly, the positioning of the binding surface regions may be based on the distribution of the magnetic particles, which is in turn based on the magnetic field generated by the magnetic element. For example, if the magnetic field has a highest magnetic flux density at the center of the reaction chamber, the binding surface may be positioned at the center of the reaction chamber. However, gravitational forces also act on the magnetic particles, which may influence the behavior of the magnetic particles during sample testing and thereby lead to result variations. This may particularly be the case when the magnetic particles are concentrated in the sample. Further, while gravitational forces cause an acceleration of the particles towards the center of the Earth, other forces causing acceleration, such as from a moving vehicle, may also cause the same effects. As such, the use of the magnetic sensor system may be limited while the system is in motion, which may limit the point-of-care use environments available. For example, the magnetic sensor system may not be reliably used in an emergency vehicle while the vehicle is in motion and/or positioned on a hill, which may delay patient care.

The effect of gravitational and other forces on the magnetic particles and resultant test unreliability may be exacerbated in sample containers that include different types of capture elements as part of the binding surface to test multiple analytes of interest. For example, when one binding surface region is used for each different analyte of interest, the position of the binding surface regions may be asymmetrical with respect to the geometry of the sample container. In these cases, the influence of gravitational forces on the magnetic particles, particularly when the sample container is not at a flat orientation, may induce variations in the distribution of the magnetic particles, leading to localized areas of higher and lower magnetic particle concentration. When the binding surface layout is asymmetric with respect to the geometry of the sample container, uneven distribution of the magnetic particles may result in one or more binding surface regions being positioned in a region of higher or lower magnetic particle distribution, which may lead to errors in the estimated concentrations of target molecules.

Thus, according to embodiments disclosed herein, a sensor system may include a sample container with one or more reaction chambers. The reaction chamber may have a detection surface that is functionalized (e.g., with antibodies) to form a binding surface. The reaction chamber further includes a spatial distribution profile of signal generating elements (e.g., magnetic particles) over the binding surface of the sample container, where the spatial distribution profile includes a gradient having a highest density along or aligned at a given reaction chamber location, such as along a central axis of the sample container and/or reaction chamber. The binding surface may be arranged with a layout that is symmetric with respect to the spatial distribution profile, such as a layout that is symmetric with respect to the central axis (e.g., the central axis is a line symmetry for the binding surface). By doing so, the regions of the binding surface may be positioned so that each region is likely to be exposed to a relatively equal proportion of the signal generating elements. Further, if the sample cartridge is subject to uneven gravitational or other forces that cause an uneven distribution of the signal generating elements, the symmetric layout of the binding surface may ensure that a binding surface region that is exposed to a relatively high proportion of the signal generating elements is balanced by a corresponding, symmetric binding surface region that is exposed to a correspondingly lower proportion of signal generating elements, thereby compensating for any gravitational or other effects causing variations in the signal generating element distribution and reducing signal variations.

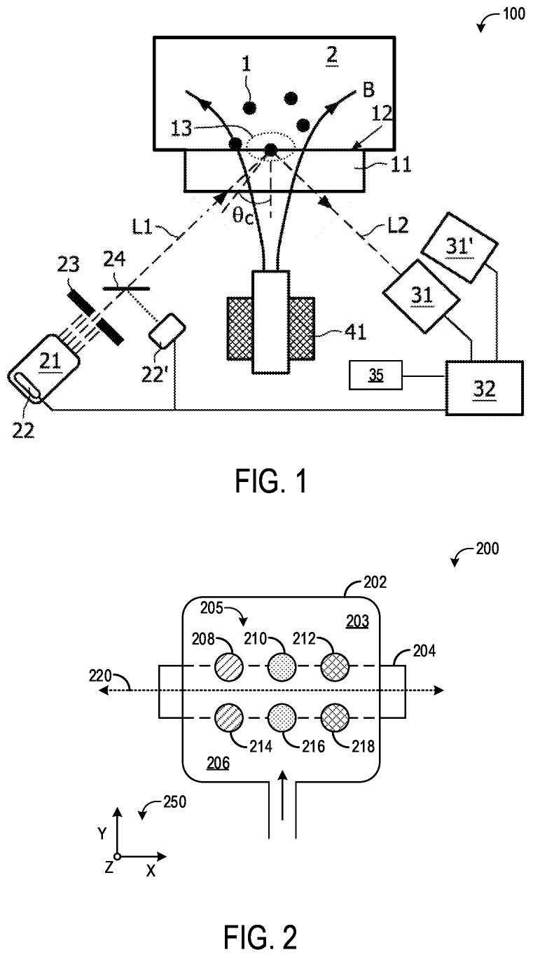

FIG. 1 schematically shows the general setup of a microelectronic sensor system 100 according to the present disclosure. The system 100 includes a carrier 11 that may for example be made from glass or transparent plastic like poly-styrene. The carrier 11 is located next to (e.g., below) a sample chamber 2 in which a sample fluid with target components to be detected (e.g. drugs, antibodies, DNA, etc.) can be provided. In some examples, the sample chamber 2 may be an interior area of a sample cartridge and the carrier 11 may form the bottom surface of the sample cartridge. In other examples, the sample chamber 2 may be an interior area of a microwell plate or other suitable container. The sample further comprises signal generating elements 1, for example superparamagnetic beads, wherein these elements 1 may be bound as labels to the aforementioned target components (for simplicity only the signal generating elements 1 are shown in FIG. 1).

The interface between the carrier 11 and the sample chamber 2 is formed by a surface referred to as a detection surface 12. This detection surface 12 may be coated with capture elements, e.g. antibodies, which can specifically bind the target components. Additional details regarding the coating of the detection surface 12 with capture elements is provided below.

The sensor system 100 comprises a magnetic field generator 41, for example an electromagnet with a coil and a core, for controllably generating a magnetic field B at the detection surface 12 and in the adjacent space of the sample chamber 2. With the help of this magnetic field B, the signal generating elements 1 can be manipulated, i.e. be magnetized and particularly be moved (if magnetic fields with gradients are used). Thus it is for example possible to attract signal generating elements 1 to the detection surface 12 in order to accelerate the binding of the associated target component to the detection surface 12.

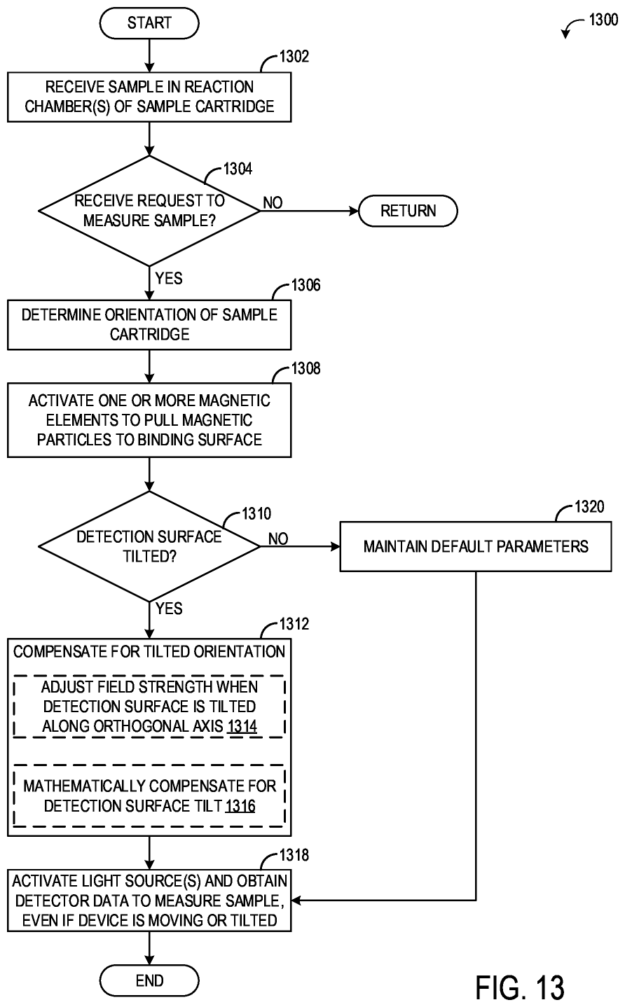

The sensor system 100 further comprises a light source 21, for example a laser or a light emitting diode (LED), that generates an input light beam L1 which is transmitted into the carrier 11. The input light beam L1 arrives at the detection surface 12 at an angle larger than the critical angle θc of total internal reflection (TIR) and is therefore totally internally reflected as an output light beam L2. The output light beam L2 leaves the carrier 11 through another surface and is detected by a light detector 31, e.g. a photodiode. The light detector 31 determines the amount of light of the output light beam L2 (e.g. expressed by the light intensity of this light beam in the whole spectrum or a certain part of the spectrum). The measurement results are evaluated and optionally monitored over an observation period by an evaluation and recording module 32 that is coupled to the detector 31. Module 32 may receive input data from the detector 31, process the input data, and output information for display on a display system and/or for storage (e.g., in a patient electronic medical record) in response to the processed input data, based on instruction or code programmed therein, corresponding to one or more routines. In particular, module 32 may be a microcomputer, including microprocessor unit, input/output ports, an electronic storage medium for executable programs and calibration values such as a read only memory chip, random access memory, keep alive memory, and a data bus. The storage medium read-only memory can be programmed with computer readable data representing instructions executable by the processor for performing the control methods for different components of FIG. 1, such as the method described below with respect to FIG. 13. Further, module 32 may be configured (e.g., execute instructions) to control the magnetic field generator 41 to provide a continuous or pulsed magnetic field when commanded, such as by controlling a current supply to the magnetic field generator 41.

In the light source 21, a commercial (λ=658 nm) laser-diode can be used. A collimator lens may be used to make the input light beam L1 parallel, and a pinhole 23 of e.g. 0.5 mm may be used to reduce the beam diameter. For accurate measurements, a highly stable light source is required. However, even with a perfectly stable power source, temperature changes in the laser can cause drifting and random changes in the output.

To address this issue, the light source may optionally have an integrated input light monitoring diode 22 for measuring the output level of the laser. The (low-pass filtered) output of the monitoring diode 22 can then be coupled to the module 32, which can divide the (low-pass filtered) optical signal from the detector 31 by the output of the monitoring diode 22. For an improved signal-to-noise ratio, the resulting signal may be time-averaged. The division eliminates the effect of laser output fluctuations due to power variations (no stabilized power source needed) as well as temperature drift (no precautions like Peltier elements needed).

In some examples, the final output of the light source 21 may be measured. As FIG. 1 coarsely illustrates, only a fraction of the laser output exits the pinhole 23. Only this fraction will be used for the actual measurement in the carrier 11, and is therefore the most direct source signal. Obviously, this fraction is related to the output of the laser, as determined by e.g. the integrated monitoring diode 22, but will be affected by any mechanical change or instability in the light path (a laser beam profile is approximately elliptical with a Gaussian profile, i.e. quite non-uniform). Thus, it is advantageous to measure the amount of light of the input light beam L1 after the pinhole 23 and/or after eventual other optical components of the light source 21. This can be done in a number of ways. For example, a parallel glass plate 24 can be placed under 45° or a beam splitter cube (e.g. 90% transmission, 10% reflection) can be inserted into the light path behind the pinhole 23 to deflect a small fraction of the light beam towards a separate input-light monitoring sensor 22′. As another example, a small mirror at the edge of the pinhole 23 or the input light beam L1 can be used to deflect a small part of the beam towards a detector.

FIG. 1 includes a second light detector 31′ that can alternatively or additionally be used to detect fluorescence light emitted by fluorescent particles which were stimulated by the evanescent wave of the input light beam L1. As this fluorescence light is usually emitted isotropically to all sides, the second detector 31′ can in principle be disposed anywhere, e.g. also above the detection surface 12. Moreover, it is of course possible to use the detector 31, too, for the sampling of fluorescence light, wherein the latter may for example spectrally be discriminated from reflected light L2.

In some examples, sensor system 100 may include a position sensor 35. The position sensor 35 may include an accelerometer, a gyroscope, and/or a geomagnetic sensor. For example, the position sensor may be configured as an inertial movement unit (IMU) including a three-axis or three-degree of freedom (3DOF) position sensor system. This example position sensor system may, for example, include three gyroscopes to indicate or measure a change in orientation of the sensor system within 3D space about three orthogonal axes (e.g., roll, pitch, and yaw). In another example, the IMU may be configured as a six-axis or six-degree of freedom (6DOF) position sensor system. Such a configuration may include three accelerometers and three gyroscopes to indicate or measure a change in location of the sensor system 100 along three orthogonal spatial axes (e.g., x, y, and z) and a change in system orientation about three orthogonal rotation axes (e.g., yaw, pitch, and roll). Output from the position sensor 35 may be sent to the module 32, in order to adjust operation of the sensor system 100 based on the orientation of the sensor system 100, in some examples.

While FIG. 1 shows a microelectronic sensor system that uses an optical detection system to measure the concentration of analyte in the sample, other mechanisms for detecting the signal generating elements bound to the binding surface are possible. For example, the signal generating elements bound to the binding surface may be detected using magneto-resistive methods, hall-sensors, coils, optical methods, imaging, fluorescence, chemiluminescence, absorption, scattering, surface plasmon resonance, Raman, sonic detection, e.g. surface-acoustic-wave, bulk acoustic wave, cantilever, quartz crystal etc., electrical detection, e.g. conduction, impedance, amperometric, redox cycling, etc.

FIG. 2 schematically shows a top-down view 200 of an example sample cartridge 202 of a magnetic sensor system, such as the sample cartridge of the microelectronic sensor system 100 of FIG. 1. Sample cartridge 202 may include a plurality of walls and a hollow interior, thereby forming a reaction chamber 203. Reaction chamber 203 is a non-limiting example of sample chamber 2 of FIG. 1. During testing, a sample (liquid including one or more analytes of interest) is loaded into the sample cartridge 202 via an inlet, which may be along the arrow shown in FIG. 2. Sample cartridge 202 includes a binding surface 205 comprising capture elements coated on a bottom surface of the sample cartridge 202 (the bottom surface of the sample cartridge is also referred to as a detection surface 206). In the example shown, sample cartridge 202 includes six binding surface regions arranged into two rows. The binding surface 205 includes a first region 208, a second region 210, and a third region 212 arranged in a first row, and a fourth region 214, a fifth region 216, and a sixth region 218 arranged in a second row. Each region may include a capture element, such as an antibody, bound to the detection surface 206. Each binding surface region may include the same capture element (e.g., the same antibody), or one or more of the binding surface regions may include different capture elements. Each binding surface region may be a discrete area, such as a spot, including a capture element fixed/coated on the detection surface at that area, and as such may also be referred to as a capture element spot or an antibody spot. In between each binding surface region, the detection surface may not include any bound capture elements, at least in some examples.

Also shown in FIG. 2 is a magnetic element 204. The magnetic element 204 is a non-limiting example of magnetic field generator 41, and thus may include an electromagnet with a coil and a core, for controllably generating a magnetic field at the detection surface 206 and in the adjacent space of the reaction chamber 203.

As will be explained in more detail below, the magnetic element 204 may be configured to generate a magnetic field having a gradient, with the highest density of the gradient (e.g., the highest magnetic flux) along a magnetic axis, which in FIG. 2 may be a central axis 220 of the sample cartridge 202. In the example shown in FIG. 2, the central axis 220 may extend along (e.g., parallel to and aligned with) a longitudinal axis of the magnetic element 204. Further, FIG. 2 includes a Cartesian coordinate system 250, and the central axis 220 extends along (e.g., is parallel to) the X axis of the coordinate system 250.

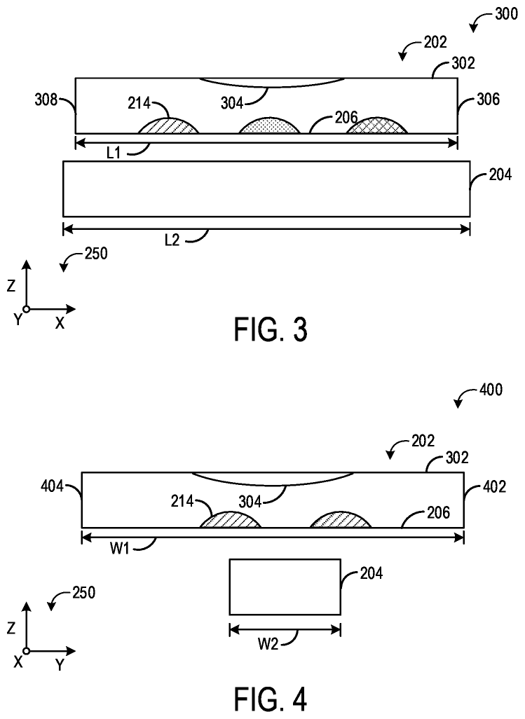

FIGS. 3 and 4 show different views of the sample cartridge 202. FIG. 3 shows a first side view 300 and FIG. 4 shows a second side view 400 of the sample cartridge 202. Each of FIGS. 3 and 4 includes the Cartesian coordinate system 250. As shown in FIG. 3, the sample cartridge 202 includes a top wall 302, a first side 306, and a second side 308. Each of the first side 306 and second side 308 extends along the Z axis of the coordinate system 250, which may be parallel to gravity and point in a direction opposite of gravity (e.g., the positive Z direction is upward, away from flat ground). As shown in FIG. 4, the sample cartridge 202 also includes a third side 402 and a fourth side 404. Also shown in FIGS. 3 and 4 is a signal generating element region 304 where dried functionalized signal generating elements, such as magnetic particles (e.g., beads), may be temporarily located. As shown, the signal generating element region 304 may be on an inner top surface (e.g., an inner surface of the top wall 302) of the sample cartridge 202, but other locations are possible and/or more than one signal generating element region may be included. When the sample is loaded into the sample cartridge 202, the dried functionalized signal generating elements may be released and mix with the sample.

As shown in FIG. 3, the sample cartridge 202 has a length L1 that extends from the first side 306 to the second side 308 along the X axis of the coordinate system 250. The magnetic element 204 has a length L2 that extends along the X axis, parallel to the longitudinal axis of the magnetic element 204. In the example shown, the length L2 of the magnetic element 204 may be as long or longer than the length L1 of the sample cartridge 202.

As shown in FIG. 4, the sample cartridge 202 has a width W1 that extends from the third side 402 to the fourth side 404 along the Y axis. In some examples, the width W1 of the sample cartridge 202 may be equal to the length L1 of the sample cartridge 202. In other examples, the width W1 may be longer or shorter than the length L1. The magnetic element 204 has a width W2 that extends along the Y axis, perpendicular to the longitudinal axis of the magnetic element 204. In the example shown, the width W2 of the magnetic element 204 is shorter than width W1 of the sample cartridge 202. Further, the magnetic element 204 may be centered with respect to the sample cartridge 202, such that a central longitudinal axis of the magnetic element 204 is aligned with a central axis of the sample cartridge 202, where the central axis of the sample cartridge may be positioned at a point equidistant between the third side 402 and the fourth side 404 and extend from the first side 306 to the second side 308. In this way, the central longitudinal axis of the magnetic element 204 may be positioned between the two rows of binding surface regions.

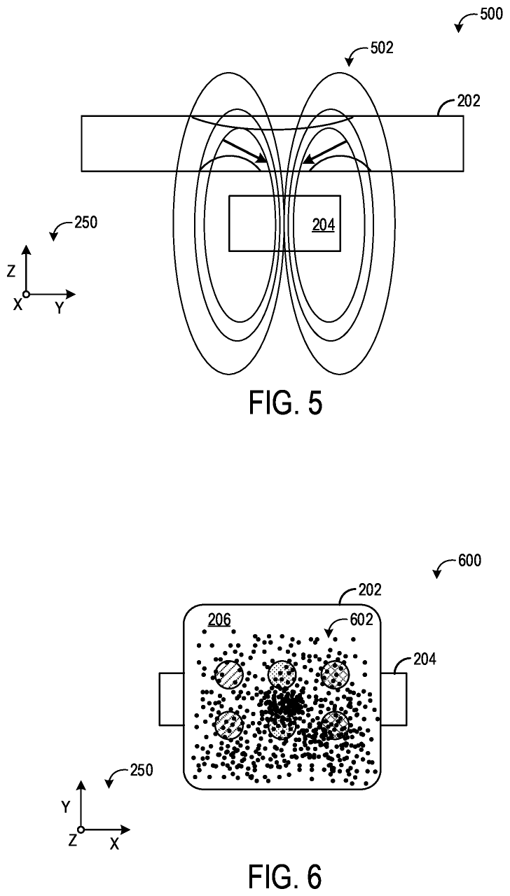

FIG. 5 shows a simplified view 500 of the sample cartridge 202 and magnetic element 204 from the same perspective as FIG. 4 and including example magnetic field lines 502 representing the magnetic field generated by the magnetic element 204. As depicted by the arrows in FIG. 5, the magnetic element 204 causes magnetic field gradients toward the center of the sample cartridge, e.g., along the central axis 220. Because the magnetic element 204 has a longer length L2 than the length L1 of the sample cartridge 202, the magnetic field gradient may be consistent along the length L1 of the sample cartridge 202 but may vary along the width W1 of the sample cartridge 202. For example, along the central axis 220, the magnetic field may have a highest flux density along an entirety of the central axis 220 from the first side 306 to the second side 308. However, the magnetic flux density may decrease from the central axis 220 to the third side 402 and from the central axis 220 to the fourth side 404.

Thus, the positioning of the sample cartridge 202 relative to the magnetic element 204 may cause a magnetic field to be generated when the magnetic element 204 is activated (e.g., current is supplied to the coil of the magnetic element 204). The magnetic field may have a gradient with a region of highest magnetic flux density positioned along the center of the sample cartridge, e.g., the central axis 220. In this way, a magnetic field gradient is formed, with the highest density of the gradient located along the central axis 220 and decreasing in each direction away from the central axis 220. After the sample is loaded into the sample cartridge 202, the signal generating elements (e.g., the magnetic particles) are released and mix with the sample. When the magnetic element 204 is activated, the magnetic particles (and any bound analyte) will be pulled to the binding surface 205 by magnetic force, and particularly be pulled toward the central axis 220, where the magnetic particles will interact with the capture elements fixed to the binding surface 205. Thus, the signal generating elements (e.g., the magnetic particles) dispersed in the sample will exhibit a spatial distribution profile over the detection surface 206, where the distribution of the signal generating elements includes a gradient of signal generating element concentration along an orthogonal axis (e.g., orthogonal to the central axis 220), as the signal generating elements may concentrate toward the location of the highest field line density/highest magnetic flux density.

Accordingly, to ensure consistent analyte analysis, particularly when more than one analyte is being tested, the binding element regions may be positioned at or in proximity to the location of the highest field line density/flux density. For example, referring back to FIG. 2, the binding surface 205 is in proximity to the central axis 220 (e.g., each region may be arranged above the magnetic element and within a threshold distance from the central axis 220). In this way, when the magnetic particles are concentrated at the binding surface 205 via a magnetic field generated by the magnetic element, the magnetic particles will concentrate at and along the binding surface 205, which will increase the likelihood that any magnetic particle/analyte complexes will interact with and bind the appropriate antibodies on the binding surface 205.

Additionally, the binding surface 205 is arranged in a symmetric manner about the central axis 220. In this way, the central axis 220 may be a line of symmetry for the binding surface 205. The symmetric arrangement of the binding surface 205 includes the binding surface regions being positioned in a symmetric manner. For example, the first region 208, the second region 210, and the third region 212 are positioned symmetrically with the fourth region 214, the fifth region 216, and the sixth region 218, respectively, with respect to the central axis 220 (e.g., a center of the first region 208 is spaced apart from the central axis 220 by an amount that is equal to an amount that a center of the fourth region 214 is spaced apart from the central axis 220). Further, the binding surface 205 may be symmetric with respect to the shape(s) of the binding surface regions. As shown, each binding surface region has the same shape and is the same size (e.g., each region may be a circle having the same diameter), but different regions may have different shapes or be of different sizes as long as the regions maintain symmetry with respect to the central axis 220. For example, the first region 208 may have the same shape as and be the same size as the fourth region 214, the second region 210 may have the same shape as and be the same size as the fifth region 216, and the third region 212 may have the same shape as and be the same size as the sixth region 218. However, the first region 208 may have a different shape and/or be of a different size than the second region 210 or the third region 212, in some examples.

Further still, the binding surface 205 may be symmetric with respect to the type(s) of capture elements included in each region. For example, the first region 208 may include a first antibody fixed to the detection surface 206, such as an anti-troponin antibody. The fourth region 214 may also include the first antibody. However, the second region 210 may include a different, second antibody, if desired, such as an anti-BNP antibody. To maintain the symmetry about the central axis 220, the fifth region 216 may also include the second antibody. In the example shown, the third region 212 may include a third antibody that is different than the first and second antibodies. The sixth region 218 may also include the third antibody.

In this way, the binding surface 205 may be arranged in a symmetric manner with respect to the central axis 220. The symmetry may include symmetry of the size, the shape, and/or the position of each region, and the symmetry may also include symmetry of the type of capture element in each region, e.g., the type of antibody in each region. By arranging the binding surface regions in a symmetric manner with respect to the location of the highest magnetic flux density, signal variations caused by gravitational effects on the sample cartridge (e.g., non-flat orientations of the detection surface 206) may be reduced, which may allow the magnetic sensor system to be used in moving vehicles, while being held in the hand of a user, etc. While the binding surface 205 is described herein as having a line of symmetry that constitutes the central axis 220, in some examples the line of symmetry of the binding surface 205 may be positioned within a threshold range of the central axis 220, such as within a certain distance of the central axis 220 (e.g., within 1 mm of the central axis 220, within 5-10% of an overall width of the detection surface or of the binding surface in a direction orthogonal to the central axis 220, etc.). Further, the line of symmetry may be parallel to the central axis 220, or the line of symmetry may be within a threshold angle of the central axis 220, such as within 5° of the extent of the central axis 220. Further, the binding surface being symmetric with respect to the size, the shape, and/or the position of each region may include the symmetric regions being within a threshold size, shape, or position of each other. For example, two symmetric regions may be considered symmetric with respect to size if a size of one of the regions is within 5-10% of the size of the other region.

FIGS. 6-8B illustrate the spatial distribution profiles of magnetic particles in the sample cartridge 202 as the sample cartridge 202 is moved into different orientations. FIG. 6 shows an example magnetic particle distribution 600 when the sample cartridge 202 is flat/not tilted and the magnetic element 204 has not been activated. Because the sample cartridge 202 is flat in the example shown in FIG. 6, the direction of gravity aligns with the Z axis and the detection surface 206 of the sample cartridge extends in parallel to both the X axis and the Y axis. When the sample cartridge 202 is filled with the sample, magnetic particles 602 in the sample cartridge 202 disperse and may at least temporarily be in a state in which the magnetic particles are highly concentrated in one or more areas of the sample cartridge 202. If the sample cartridge 202 orientation is changed (e.g., tilted to the left or right, or top or bottom), gravity or other forces will induce movement of the higher mass density fluid in different directions, causing the magnetic particles to distribute in different ways, but in all cases inhomogenously within the fluid. As a result, magnetic particle concentrations above each binding surface region may be different, and consequently cause reactions occurring at each binding surface region to reach different results than they would if the system is on a flat surface (e.g., more or fewer magnetic particles will bind).

By using the sample cartridge and magnetic element of the present disclosure, the magnetic particle distribution over the binding surface of the sample cartridge 202 can be aligned at a given location, e.g., along the central axis 220. By arranging the binding surface regions to be symmetric with respect to the center of alignment of the magnetic particle distribution, the magnetic sensor system may be configured to prevent signal variations caused by different orientations of the sample cartridge 202.

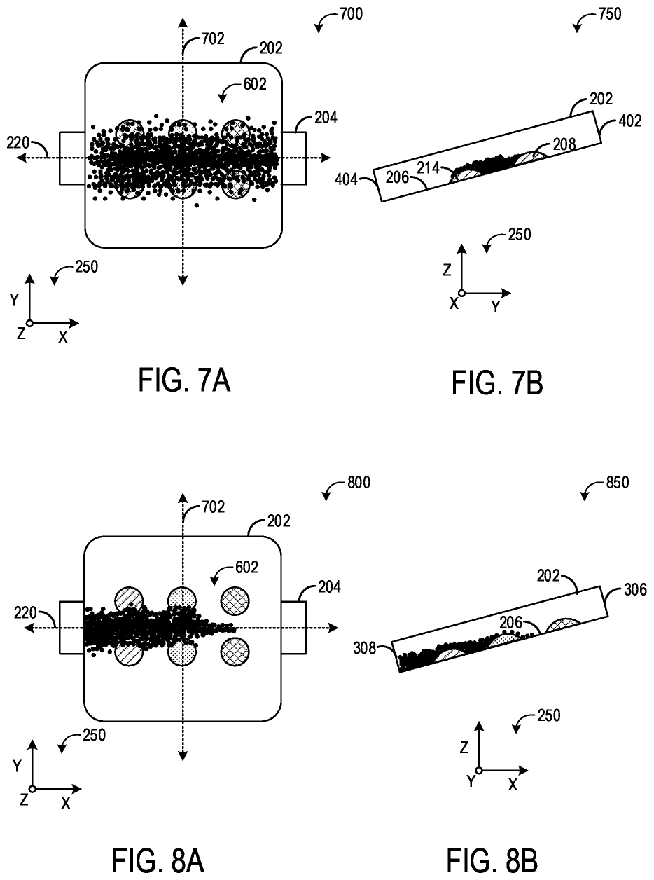

For example, FIG. 7A shows another example magnetic particle distribution 700 when the magnetic element 204 is activated and thus generating a magnetic field and the sample cartridge 202 is in a default, flat orientation. Because the highest magnetic flux density extends along the central axis 220, the magnetic particles 602 are dispersed relatively evenly along the central axis 220 but form a gradient along an orthogonal axis 702, which is orthogonal to the central axis 220. The concentration of the magnetic particles is the highest at and along the central axis 220 and the concentration of the magnetic particles decreases in both directions along the orthogonal axis 702 (e.g., away from the central axis 220). In this way, the gradient in the spatial distribution profile of the plurality of signal generating elements is such that that the spatial distribution profile has a maximum concentration and then decreases in both directions along the orthogonal axis. As a result of the symmetric arrangement of the binding surface 205 (e.g., the binding surface having an axis of symmetry that is orthogonal to the orthogonal axis 702), each binding surface region may be exposed to the same relative proportion of magnetic particles and signal variations among the binding surface regions may be due to differences in analyte concentration in the sample and not due to varying concentrations of the magnetic particles relative to the binding surface.

FIG. 7B shows another example magnetic particle distribution 750 when the sample cartridge 202 in a first tilted orientation. In the first tilted orientation, the detection surface 206 of the sample cartridge 202 is tilted along the Y axis, such that the fourth side 404 is closer to ground than the third side 402, and is not tilted along the X axis (where the central axis 220 is parallel to the X axis). The tilted orientation of the sample cartridge 202 may result in a higher concentration of the magnetic particles being positioned over/at the bottom row of binding surface regions (e.g., including the fourth region 214) than the top row of binding surface regions (e.g., including the first region 208). Because of the symmetric arrangement of the binding surface 205 relative to the central axis 220, any increased binding of magnetic particles at the fourth region 214, for example, may be balanced by a corresponding decrease in binding of magnetic particles at the first region 208. In some examples, signals measured at each binding surface region for a given analyte/capture element may be averaged to generate an overall signal that is equivalent to the signal that would be generated if the sample cartridge 202 were flat. As a result, the change in orientation of the sample cartridge 202 may not result in signal variations.

Thus, the binding surface of the sample container may arranged so that a center of mass of the spatial distribution profile of the signal generating elements is above the binding surface, rather than to the side of the binding surface. For example, a fraction of signal generating elements from the spatial distribution profile which overlaps the binding surface may be 50% or more, 30% or more, 10% or more, 5% or more. Further, the center of mass of the spatial distribution profile of the signal generating elements distribution mapped on a plane of the binding surface may be within the binding surface. That is, magnetic element and the binding surface may be arranged so that the gradient of signal generating elements that is induced upon activation of the magnetic element is located above and centered over the binding surface. However, the spatial distribution profile may include a second gradient that is centered over a second axis, orthogonal to the central axis 220 described above, during some conditions. For example, FIG. 8A shows another example magnetic particle distribution 800 when the magnetic element 204 is activated and thus generating a magnetic field and the sample cartridge 202 is in a second tilted orientation 850, as shown in FIG. 8B. In the second tilted orientation 850, the detection surface 206 of the sample cartridge 202 is tilted along the X axis (where the central axis 220 is parallel to the X axis), such that the second side 308 is closer to ground than the first side 306, and the sample cartridge 202 is not tilted along the Y axis.

As appreciated from FIG. 8A, the magnetic element 204 attracts the magnetic particles 602 to the central axis 220. However, because detection surface 206 of the sample cartridge 202 is tilted along the central axis 220, the magnetic particles 602 are concentrated at one side of the sample cartridge 202 (e.g., at the second side 308), due to the non-even effect of gravity on the tilted cartridge. As such, the binding surface may be exposed to different concentrations of magnetic particles and signal variations may occur. For example, the third region and the sixth region, which may each include a third capture element specific to a third analyte, may be exposed to fewer magnetic particles than the second region and the fifth region (which may each include a second capture element specific to a second analyte), and the first region and the region (which may each include a first capture element specific to a first analyte). This may result in a measurement of the third analyte that is artificially lower than the actual concentration of the third analyte in the sample. To reduce or prevent this signal variation, the binding surface may be arranged in a manner so as to be symmetric with respect to the orthogonal axis 702, as explained below.

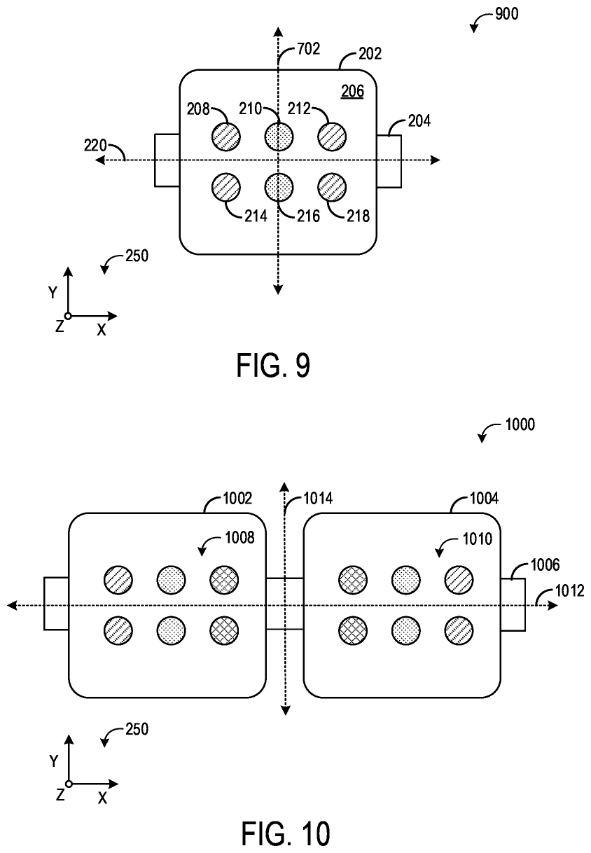

FIG. 9 shows another view 900 of the sample cartridge 202. In the view 900 shown in FIG. 9, the arrangement of the plurality of capture element spots is symmetric to both the central axis 220 and the orthogonal axis 702. The orthogonal axis 702 extends parallel to the Y axis and perpendicular to the central axis 220. The orthogonal axis 702 may be aligned with a center of the sample cartridge 202 and thus may be positioned equidistant to both the first side 306 and the second side 308. The symmetric arrangement of the binding surface includes the binding surface regions being arranged symmetrically with respect to size, position, shape, and capture element type. For example, the first region 208 has a first size and a first shape and also is comprised of a first antibody. The second region 210 has a second size and a second shape and also is comprised of a second antibody. The second shape may be mirror symmetric with respect to the orthogonal axis 702.

To achieve the mirror symmetry across both the central axis 220 and the orthogonal axis 702, the third region 212 is arranged symmetric to the first region 208 with respect to the orthogonal axis 702. As such, the third region 212 has the first size, the first shape, and is comprised of the first antibody. Further, the third region 212 is aligned along a common axis (parallel to the X axis) with the first region 208, and the first region 208 and the third region 212 are each spaced apart from the orthogonal axis 702 by the same amount. The fourth region 214 is also arranged symmetric to the first region 208 with respect to the central axis 220. As such, the fourth region 214 has the first size, the first shape, and is comprised of the first antibody. Further, the fourth region 214 is aligned along a common axis (parallel to the Y axis) with the first region 208, and the first region 208 and the fourth region 214 are each spaced apart from the central axis 220 by the same amount. The fifth region 216 is arranged symmetrically with the second region 210 with respect to the central axis 220. As such, the fifth region 216 has the second size, the second shape, and is comprised of the second antibody. Further, the fifth region 216 is aligned along the orthogonal axis 702 with the second region 210, and the second region 210 and the fifth region 216 are each spaced apart from the central axis 220 by the same amount. The sixth region 218 is arranged symmetric to the third region 212 with respect to the central axis 220 and is also arranged symmetric to the fourth region 214 with respect to the orthogonal axis 702. Thus, the sixth region has the first shape, the first size, and is comprised of the first antibody.

In the example shown, the first size may be equal to the second size and the first shape may be the same as the second shape, while the first antibody may be different than the second antibody (e.g., the first antibody may be anti-troponin and the second antibody may be anti-BNP). However, other shapes, sizes, and antibody arrangements are possible as long as the symmetry across both the central axis 220 and the orthogonal axis 702 is maintained. For example, the second size may be larger than the first size (e.g., the diameter of the circles may be larger for the second and fifth capture element spots) and/or the second shape may be different than the first shape (e.g., the second shape may be a square rather than a circle).

When the binding surface is arranged in a symmetric manner to have a first line of symmetry that extends along and is aligned with a magnetic axis and a second line of symmetry that is orthogonal to the magnetic axis (and where both the magnetic axis and the orthogonal axis are lines of symmetry for the geometry of the reaction chamber), any changes in orientation of the sample cartridge that result in localized high concentrations of magnetic particles may be compensated for. For example, if the sample cartridge 202 shown in FIG. 9 is tilted along the X axis as shown in FIG. 8B, higher magnetic particle concentration at the first region 208 and the fourth region 214 may be compensated for by a correspondingly lower magnetic particle concentration at the third region 212 and sixth region 218.

Additionally, as shown in FIG. 10, more than one reaction chamber may be included in a sample cartridge and/or more than one sample cartridge may be present in a sensor system. When two or more chambers and/or cartridges are present, the symmetry across the axes described above may be maintained by moving the orthogonal axis to be between the chambers or cartridges.

FIG. 10 shows a schematic view 1000 of a dual chamber sensor system including a first sample cartridge 1002 and a second sample cartridge 1004. The first sample cartridge 1002 may share a common inlet with and/or otherwise be fluidly coupled to the second sample cartridge 1004. In other examples, each cartridge may include a respective inlet and may be maintained fluidly separate. The dual chamber sensor system includes a magnetic element 1006, which in the example shown is positioned beneath the sample cartridges. The magnetic element 1006 may be similar to the magnetic element 204 and thus may generate a magnetic field having a gradient with a highest magnetic flux density along a center/longitudinal axis, herein the central axis 1012. While one magnetic element is shown in FIG. 10, more than one magnetic element may be present (e.g., a magnetic element identical to magnetic element 204 may be positioned under each sample cartridge rather than one continuous magnetic element).

Each sample cartridge may include a binding surface. For example, the first sample cartridge 1002 may include a first binding surface 1008 and the second sample cartridge 1004 may include a second binding surface 1010. Each binding surface may be similar to the binding surface described above, e.g., include one or more regions each having a capture element fixed to the detection surface of the sample cartridge.

The first binding surface 1008 and the second binding surface 1010 may be arranged to have collective symmetry across the central axis 1012 and an orthogonal axis 1014 positioned equidistant from the first sample cartridge 1002 and the second sample cartridge 1004. For example, as shown, three different types of antibodies are present in the binding surfaces, with a first antibody (schematically depicted in striped lines) arranged in a first column of the first binding surface 1008 and a last column of the second binding surface 1010, a second antibody (schematically depicted in dots) arranged in each middle column of the binding surfaces, and a third antibody (schematically depicted in cross-hatched lines) arranged in a last column of the binding surface 1008 and a first column of the second binding surface 1010. The binding surface regions may be symmetric in size and shape across each of the central axis 1012 and the orthogonal axis 1014.

As described above, the binding surface 205 is described herein as having one or more additional lines of symmetry that constitute the orthogonal axis 702 and/or an axis between two reaction chambers, but in some examples the lines of symmetry of the binding surface 205 may be positioned within a threshold range of the respective axis, such as within a certain distance of the axis (e.g., within 1 mm of the axis, within 5-10% of an overall width of the detection surface or of the binding surface in a direction orthogonal to the axis, etc.). Further, the lines of symmetry may be parallel to the respective axis, or the lines of symmetry may extend within a threshold angle of the axis, such as within 5° of the extent of the axis.

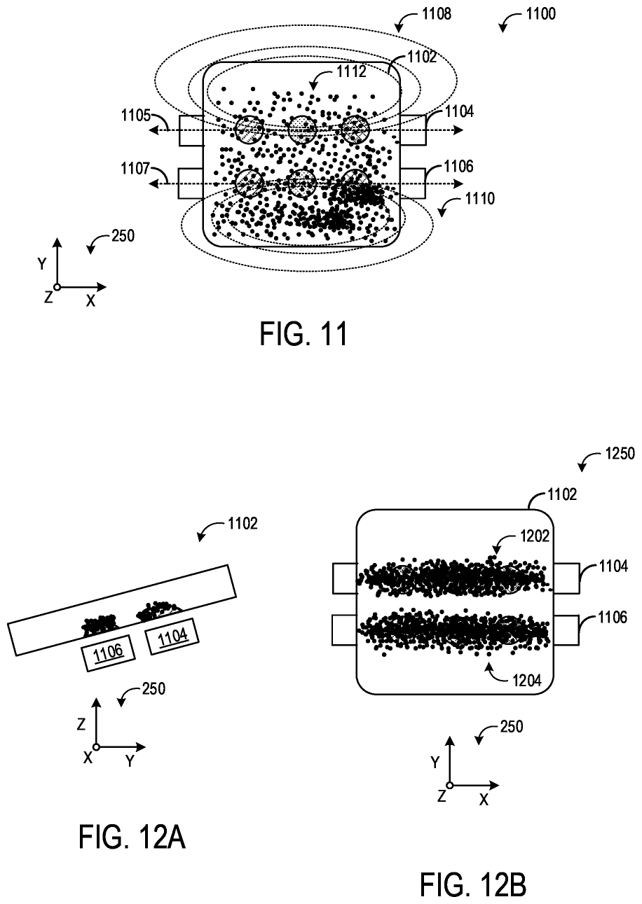

In some examples, two or more magnetic elements may be included in proximity to a sample cartridge, which may result in two or more areas of highest magnetic force and thus two or more gradients of signal generating elements (e.g., magnetic particles). In such examples, the binding surface regions may be arranged in two or more groups so as to overlap/align with each area of highest magnetic force, and each group may be arranged symmetrically with respect to a respective area. For example, FIG. 11 schematically shows a view 1100 of a dual-magnet sensor system including a sample cartridge 1102, a first magnetic element 1104, and a second magnetic element 1106. The sample cartridge 1102 may be similar to the sample cartridge 202 and thus may include a binding surface arranged on a bottom binding surface of the sample cartridge. Each magnetic element may extend with a longitudinal axis that is parallel to the X axis, and each magnetic element may be longer than the length of the sample cartridge 1102.

The binding surface may include regions arranged into two groups, e.g., a first row and a second row. The first row of binding surface regions may be arranged along a first magnetic axis 1105 that comprises an axis of highest magnetic flux density generated by the first magnetic element 1104. The second row of binding surface regions may be arranged along a second magnetic axis 1107 that comprises an axis of highest magnetic flux density generated by the second magnetic element 1106. The first row of binding surface regions may be arranged symmetrically with respect to the first magnetic axis 1105 and the second row of binding surface regions may be arranged symmetrically with respect to the second magnetic axis 1107.

As shown in FIG. 11, a plurality of magnetic particles 1112 may be released into the sample cartridge 1102 when a sample is introduced and the magnetic particles may disperse in a non-homogenous manner. To expedite reaction time and assist in a more even distribution of magnetic particles relative to the binding surface, the first magnetic element 1104 and/or the second magnetic element 1106 may be activated to generate a respective magnetic field along a corresponding magnetic axis. Further, the strength of each magnetic field may be controlled based on the orientation of the sample cartridge, to further reduce or prevent the effects of the orientation of the sample cartridge on the distribution of the magnetic particles. For example, if the sample cartridge 1102 were to be tilted along the Y axis as shown by FIG. 12A, the magnetic elements may be controlled (e.g., adjusted) to have different magnetic field strengths to compensate for the tilted orientation. As shown in FIG. 11, the first magnetic element 1104 may be controlled to have a larger magnetic field strength than the second magnetic element 1106 when the sensor system (including the sample cartridge and the magnetic elements) is tilted along the direction shown in FIG. 12A (such that the first magnetic element 1104 is farther from ground than the second magnetic element 1106). The differences in the magnetic field strengths are shown schematically by example magnetic field lines 1108 and 1110.

As a result, and as shown in FIG. 12B, the magnetic particles may distribute into two gradients, a first gradient 1202 and a second gradient 1204. The first gradient 1202 may be centered along the first magnetic axis 1105 and the second gradient 1204 may be centered along the second magnetic axis 1107. The differential magnetic field strengths may help compensate for the effects of gravity and other forces when the cartridge is tilted and/or in motion, so that a more even distribution of magnetic particles between the two magnetic axes is achieved.

While a sample cartridge configured to be positioned in a sensor device or sensor system has been described herein, it is to be appreciated that the sample cartridge may be any suitable container that is coated with two or more binding surface regions on a detection surface thereof and configured to house a sample mixed with signal generating elements, such as magnetic particles. For example, the sample cartridge may not be enclosed as described herein but may instead lack a top wall, or the sample cartridge may be in the form a plate including one or more wells. As such, the sample cartridge(s) described above with respect to FIGS. 2-12B may be referred to as a sample container, which may include a cartridge, a plate, a multi-well plate, or virtually any other structure capable of housing a sample and having a binding surface as described herein.

FIG. 13 is a flow chart illustrating a method 1300 for testing a sample with a sensor system, such as sensor system 100. Method 1300 may be executed at least partially by a computing system such as evaluation and recording module 32 of sensor system 100, according to instructions stored in memory thereof. At 1302, a sample is received in a reaction chamber of the sensor system. The sample may include a biological fluid, such as blood, saliva, etc., which may be mixed with reagents, buffers, water, etc. The sample may be introduced via a sample inlet and may flow into the reaction chamber. The reaction chamber may comprise an interior of a sample container, such as sample cartridge 202. Thus, the sample may mix with signal generating elements in the reaction chamber. The sample container may include a binding surface including one or more regions of capture elements coated on a detection surface of the sample container. In some examples, a sample may be received in more than one reaction chamber of the sensor system (e.g., the sample container may include two reaction chambers, or two sample cartridges may be present in the sensor system).

At 1304, method 1300 determines if a request has been received to measure the concentration of one or more analytes in the sample. The request may be received via user input entered to the sensor system (e.g., user selection of a “start” button on a user interface of the sensor system). If a request has not been received, method 1300 returns and continues to monitor for a sample measurement request being received.

If a request to measure the sample has been received, method 1300 proceeds to 1306 to optionally determine the orientation of the detection surface of the sample container. If present in the sensor system, a position sensor (such as position sensor 35) may output information usable to determine an orientation of the sensor system and hence the sample cartridge. The orientation may be determined relative to a direction of gravity. For example, based on the output of the position sensor, the sensor system may determine if the detection surface (which in a default, flat orientation may extend in a horizontal plane perpendicular to gravity and parallel to flat ground) is flat or if the detection surface is tilted with respect to gravity. The output of the position sensor may further be used to determine the axis and direction of the tilt. In some examples, the output from the position sensor may be used to determine if the sensor system and hence the sample cartridge is accelerating.

At 1308, one or more magnetic elements of the sensor system are activated in order to attract the signal generating elements, which may be magnetic particles, to the binding surface of the sample cartridge. The one or more magnetic elements may include one or more magnetic elements, such as magnetic element 204, that generate a magnetic field with a gradient centered along a single magnetic axis or a single point, or the one or more magnetic elements may include two or more magnetic elements, such as first magnetic element 1104 and second magnetic element 1106, that generate multiple magnetic fields with gradients centered along multiple magnetic axes or points. The magnetic element(s) may be activated to generate a continuous or a pulsed magnetic field. Further, in some examples, the generated magnetic field may be pulsed or modulated so as to perform one or more magnetic washes after pulling the magnetic particles to the binding surface.

The magnetic particles are pulled to the binding surface so that the magnetic particles have a distribution at the binding surface that is centered along the magnetic axis (or axes when more than magnetic element is present) or the magnetic point(s) and have a gradient that extends along an axis orthogonal to the magnetic axis. Because the binding surface includes a plurality of regions arranged symmetrically with respect to the magnetic axis or point, the magnetic particles may distribute in an equal manner with respect to the binding surface, and thus signal variations due to inhomogenous magnetic particle distribution may be reduced or avoided.

At 1310, method 1300 optionally determines if the detection surface is tilted, based on the orientation of the sample cartridge determined at 1306 (e.g., based on the output of the positon sensor). If the detection surface is tilted, method 1300 proceeds to 1312 to optionally compensate for the tilted orientation of the detection surface. In one example, compensating for the tilted orientation may include adjusting the magnetic field strength generated by one or more of the magnetic elements based on the orientation of the binding surface, as indicated at 1314. Adjusting the magnetic field strength may include adjusting the magnetic field strength when the binding surface is tilted along an orthogonal axis, at least in some examples. The orthogonal axis may be perpendicular to the magnetic axis of the magnetic element. For example, referring to FIGS. 11 and 12A, the magnetic elements may be positioned to generate two parallel magnetic axes (axes 1105 and 1107) that extend along the X axis of the coordinate system 250, and the orthogonal axis may extend along the Y axis. Tilting along the orthogonal axis may result in one side of the sample container being closer to ground than another, opposite side of the sample container, in a plane perpendicular to the magnetic axis. Further, adjusting the magnetic field strength based on the orientation may include adjusting the field strength generated by one or more of the magnetic elements so that the overall magnetic field gradient induced in the sample cartridge increases in magnetic field line density/magnetic flux density along a direction opposite of the tilt. For example, the relative magnetic field strengths of two magnetic elements may be adjusted so that the magnetic element that is vertically higher than the other magnetic element has a higher field strength, to overcome the additional gravitational forces at the portion of the binding surface closer to ground. Referring to FIG. 12A, this adjustment may include increasing the magnetic field strength generated by the first magnetic element 1104 relative to the magnetic field strength generated by the second magnetic element 1106.

In some examples, as indicated at 1316, compensating for the tilted orientation may include mathematically compensating for the detection surface tilt during the processing of the detection unit output to determine the concentration of one or more analytes, as explained in more detail below. For example, the signal that is detected from the signal generating elements may be weighted for some binding surface regions relative to other binding surface regions based on the orientation, e.g., the signal from a binding surface region that is lower to ground than another binding surface region may be given a lower weight than the signal from the other binding surface region.

If method 1300 determines that the detection surface is not tilted, method 1300 proceeds to 1320 to maintain default parameters, which may include maintaining equal magnetic field strengths, e.g., each magnetic element may be controlled to generate a magnetic field of the same strength, and/or weighting the signals from each binding surface equally (or at least not weighting the signals based on the orientation of the detection surface). While compensation for device orientation is described with respect to FIG. 13 as being performed in response to an orientation of the detection surface, in some examples, the compensation may be performed as described herein in response to an acceleration of the detection surface of the sample container, e.g., the magnetic field strength may be adjusted in response to the output from the position sensor indicating that the detection surface is accelerating (e.g., due to the sample container/sensor system being positioned in a moving vehicle or on a moving cart).

At 1318, the sample is measured even if the sensor system is moving or tilted, by activating one or more light sources, such as light source 21, and obtaining detector data from one or more detectors, such as detector 31. In some examples, the light source(s) may be activated and detector data obtained during the binding of the magnetic particles to the binding surface and washing, or the light source(s) may be activated and detector data obtained only once binding and washing is complete. In some examples, the light source may be configured so that the signal generating elements are detected using frustrated total internal reflectance (fTIR). Further, the light source and detection unit may be configured such that only signal generating elements within a certain threshold distance of the detection surface are measured, such as within 100 nm of the detection surface. As such, when referring to the spatial distribution of the signal generating elements herein, the distribution may be limited to only those signal generating elements that are within 100 nm (or another suitable distance, such as 200 nm) of the detection surface. The obtained detector data may be processed to determine a concentration of one or more analytes of interest in the sample, and the determined concentration(s) or concentration signal(s) may be output for display and/or saved in memory. For example, the concentration of the analyte may be computed using a calibration curve to convert the measured amount of bound signal generating elements into a concentration of the analyte. The calibration curve (or formula, or equation) may be stored in the memory of the sensor system (e.g., the evaluation and recording module 32), and the values/parameters for the calibration curve or formula may be stored onto an RFID tag of the sensor system. The calibration parameters (e.g., the calibration curve or formula, including constants of the formula) may be determined after manufacturing by testing a series of cartridges with reference samples, e.g., samples containing different concentrations of analyte, distributed over the reportable range for the test. The test data is then subsequently analyzed by fitting (e.g., a least squares regression) the data using a mathematical formula. The resulting fit parameters are then written onto the RFID tag of the device. Method 1300 then ends.

Thus, a sensor system, such as the sensor system described above, may include a measurement device to measure an amount of bound signal generating elements and compute a concentration of an analyte in a sample. The bound signal generating elements may be signal generating elements (e.g., magnetic particles) that are bound to a capture element coated on a detection surface of a sample cartridge containing the sample, either directly or indirectly via the analyte. As described herein, the capture elements may be coated on the detection surface in a symmetric layout with respect to a gradient of the signal generating elements, to thereby form a binding surface on the detection surface. The arrangement of the binding surface (e.g., the symmetric arrangement with at least one axis of symmetry that is orthogonal to an axis defining the gradient of the signal generating elements), wherein the at least one axis of symmetry of the binding surface together with the gradient causes a number of signal generating elements of the plurality of signal generating elements that overlap the binding surface to remain constant in the presence of external forces such as gravity. In some examples, the amount of bound signal generating elements are measured by the measurement device using fTIR. Further, an orientation of the sample cartridge may be measured and used to mathematically compensate for the measured amount of bound signal generating elements when computing the concentration of the analyte.

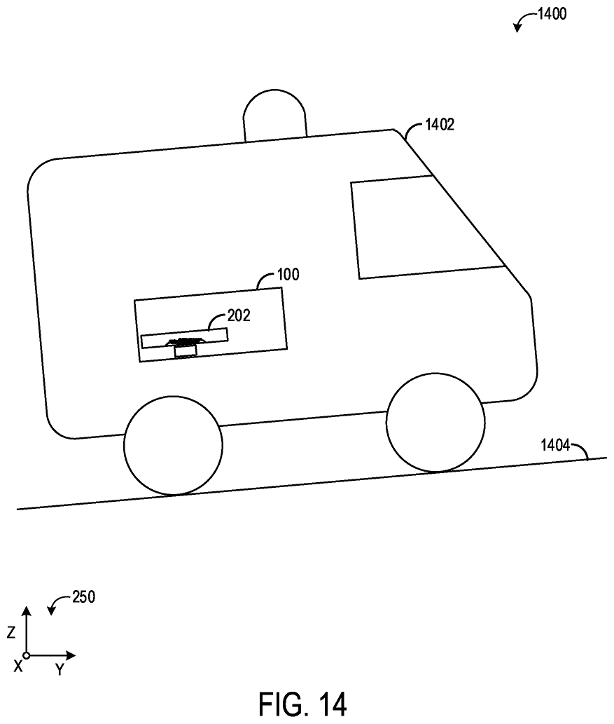

FIG. 14 shows an example 1400 of a sensor system according to the disclosure in use while in a moving vehicle. For example, a vehicle 1402 may be traveling on a surface 1404, which in the example shown is inclined along the Y axis. Sensor system 100 including sample cartridge 202 may be present in the vehicle 1402 and used to test a sample while the vehicle 1402 is moving (and particularly while the vehicle 1402 is accelerating or decelerating) and/or positioned on an incline or decline. Because of the arrangement of the binding surface of sample cartridge 202, the uneven gravitational effects to which the sample cartridge 202 is subject may be compensated and signal variations may be reduced.

The technical effect of symmetrically arranging a binding surface with respect to a gradient of signal generating elements of a sample cartridge of a sensor system is that signal variations induced by uneven gravitational effects may be reduced, particularly when more than one analyte is being tested.

The disclosure also provides support for a sensor system, comprising: a sample container configured to receive a sample containing an analyte to be tested, the sample container comprising: a detection surface, and a plurality of signal generating elements in the sample container, wherein the detection surface comprises a binding surface, which has been partially functionalized with capture elements that can bind, directly and/or indirectly, the analyte and/or the plurality of signal generating elements, wherein the plurality of signal generating elements have a spatial distribution profile over the detection surface, wherein the spatial distribution profile has a gradient along a first axis, and wherein the binding surface has an axis of symmetry that is orthogonal to the first axis. In a first example of the system, the sample container has at least one axis of symmetry, which also constitutes the axis of symmetry of the binding surface. In a second example of the system, optionally including the first example, the axis of symmetry of the binding surface is a first axis of symmetry, wherein the binding surface has a second axis of symmetry orthogonal to the first axis, wherein the sample container has two axes of symmetry, orthogonal to each other, which also constitute the first and second axes of symmetry of the binding surface. In a third example of the system, optionally including one or both of the first and second examples, the system further comprises: a magnetic field generation component, able to generate a magnetic gradient within the sample container. In a fourth example of the system, optionally including one or more or each of the first through third examples, the magnetic gradient within the sample container causes the plurality of signal generating elements to move towards the first axis of symmetry of the binding surface. In a fifth example of the system, optionally including one or more or each of the first through fourth examples, the second axis of symmetry of the binding surface together with the magnetic gradient causes a number of signal generating elements of the plurality of signal generating elements overlapping the binding surface to remain constant in the presence of external forces such as gravity. In a sixth example of the system, optionally including one or more or each of the first through fifth examples, the spatial distribution profile of the plurality of signal generating elements also has a second gradient along a second axis which is orthogonal to said first axis. In a seventh example of the system, optionally including one or more or each of the first through sixth examples, the binding surface is also symmetric along a third axis that is orthogonal to said second axis. In an eighth example of the system, optionally including one or more or each of the first through seventh examples, the gradient in the spatial distribution profile of the plurality of signal generating elements is such that that the spatial distribution profile has a maximum concentration and then decreases in both directions along the first axis. In a ninth example of the system, optionally including one or more or each of the first through eighth examples, a center of mass of the spatial distribution profile of the plurality of signal generating elements is above the binding surface. In a tenth example of the system, optionally including one or more or each of the first through ninth examples, a fraction of the spatial distribution profile which overlaps the binding surface is 50%/o or more, 30% or more, 10% or more, or 5% or more. In an eleventh example of the system, optionally including one or more or each of the first through tenth examples, the system further comprises: a measurement device to measure an amount of bound signal generating elements and compute a concentration of the analyte. In a twelfth example of the system, optionally including one or more or each of the first through eleventh examples, the concentration of the analyte is computed using a calibration curve to convert the measured amount of bound signal generating elements into a concentration of the analyte. In a thirteenth example of the system, optionally including one or more or each of the first through twelfth examples, the amount of bound signal generating elements are measured by the measurement device using fTIR. In a fourteenth example of the system, optionally including one or more or each of the first through thirteenth examples, the spatial distribution profile of the plurality of signal generating elements only includes signal generating elements within 100 nm of the detection surface. In a fifteenth example of the system, optionally including one or more or each of the first through fourteenth examples, an orientation of the sample cartridge is measured and used to mathematically compensate for the measured amount of bound signal generating elements when computing the concentration of the analyte. In a sixteenth example of the system, optionally including one or more or each of the first through fifteenth examples, the binding surface includes a first region which has been functionalized with a first type of capture element and a second region which has been functionalized with a second type of capture element, wherein the first capture element can bind, directly and/or indirectly, the analyte, and the second capture element can bind, directly and/or indirectly, a second, different analyte.

In another example of the system, optionally including one or more or each of the previously described examples, the system may be used to carry out a method including receiving a sample containing an analyte to be tested in a sample container of the sensor system, the sample container including a detection surface and a plurality of signal generating elements in the sample container, wherein the detection surface comprises a binding surface, which has been partially functionalized with capture elements that can bind, directly and/or indirectly, the analyte and/or the plurality of signal generating elements; generating a spatial distribution profile of the plurality of signal generating elements over the detection surface, wherein the spatial distribution profile has a gradient along a first axis, wherein the binding surface has an axis of symmetry that is orthogonal to the first axis; and measuring an amount of bound signal generating elements bound to the binding surface and computing a concentration of the analyte in the sample based on the amount of bound signal generating elements.

In another representation, a magnetic sensor system includes a first sample cartridge configured to receive a first sample to be tested, a second sample cartridge configured to receive the first sample or a second sample to be tested; at least one electromagnetic unit configured to produce a magnetic field at a first binding surface of the first sample cartridge and at a second binding surface of the second sample cartridge, the magnetic field having a magnetic field gradient with a highest density of magnetic field lines along a first axis; and wherein the first binding surface and the second binding surface are arranged in a symmetric manner with respect to the first axis and a second axis, orthogonal to the first axis.

In another representation, a sample cartridge for a magnetic sensor system includes a plurality of walls forming a sample chamber configured to receive a sample to be tested and a plurality of capture element spots arranged on a detection surface of the sample cartridge in a symmetric manner with respect to a first axis, where the first axis is configured to align with a magnetic axis of a magnetic element of the magnetic sensor system when the sample cartridge is loaded in the magnetic sensor system. The magnetic element is configured to produce a magnetic field at the binding surface of the sample cartridge, the magnetic field having a magnetic field gradient with a highest density of magnetic field lines along the magnetic axis.

In another representation, a method for a magnetic sensor system includes adjusting a field strength of one or more magnetic elements of the magnetic sensor system based on orientation of a binding surface of a sample container of the magnetic sensor system. In a first example of the method, the magnetic sensor system includes a first magnetic element and a second magnetic element and the binding surface extends across a first plane, and wherein adjusting the field strength comprises: if an entirety of the first plane is perpendicular to gravity, adjusting the field strength so that the first and second magnetic elements generate equal field strengths; and if the first plane is tilted with respect to gravity, adjusting the field strength of one or both of the first and second magnetic elements so that the first and second magnetic elements generate unequal field strengths. In a second example of the method, optionally including the first example, adjusting the field strength of one or both of the first and second magnetic elements so that the first and second magnetic elements generate unequal field strengths comprises generating an overall magnetic field gradient at the binding surface that increases in magnetic flux density in a direction opposite of gravity. In a third example of the method, optionally including one or both of the first and second examples, adjusting the field strength comprises adjusting the field strength to pull a plurality of magnetic particles mixed with a sample loaded in the sample container to the binding surface of the sample container along a magnetic axis, the magnetic axis forming a line of symmetry for the binding surface. In a fourth example of the method, optionally including one or more or each of the first through third examples, the binding surface includes a first capture element region specific to a first analyte and a second capture element region specific to a second analyte. In a fifth example of the method, optionally including one or more or each of the first through fourth examples, the field strength is adjusted in response to a request to measure the sample loaded into the sample container. In a sixth example of the method, optionally including one or more or each of the first through fifth examples, the method further includes measuring the sample by directing light onto the binding surface and detecting light reflected from the binding surface.

In another representation, a magnetic sensor system includes a sample container configured to receive a sample to be tested, the sample container comprising a reaction chamber comprising a binding surface that can bind magnetic particles directly or via an analyte from the sample; and a magnetic element configured to produce a magnetic field at the binding surface of the sample container, the magnetic field having a magnetic field gradient with a highest density along a first axis that crosses through the reaction chamber, wherein the binding surface is arranged in a symmetric manner with respect to the first axis. In a first example of the system, the binding surface is defined by two or more regions, where a first capture element is present in a first region of the at least two regions and a second capture element is present in a second region of the at least two regions, and wherein the first region and the second region are arranged on a same side of the first axis. In a second example of the system, optionally including the first example, the binding surface is defined by a first region arranged on a first side of the fist axis and a second region arranged on a second, opposite side of the first axis, and the first region is symmetric to the second region with respect to a size, a shape, a position, and a type of capture element present in each of the first region and the second region. In a third example of the system, optionally including one or both of the first and second examples, the binding surface is arranged in a symmetric manner with respect to a second axis, orthogonal to the first axis, wherein the second axis crosses a center of mass of the one or more reaction chambers. In a fourth example of the system, optionally including one or more or each of the first through third examples, the first axis is parallel to a longitudinal axis of the magnetic element. In a fifth example of the system, optionally including one or more or each of the first through fourth examples, the system further includes a plurality of functionalized magnetic particles arranged in the sample container. In a sixth example of the system, optionally including one or more or each of the first through fifth examples, the system further includes a detection unit for detecting functionalized magnetic particles bound to the binding surface and a processing unit configured to compute a concentration of the analyte based on output from the detection unit. In a seventh example of the system, optionally including one or more or each of the first through sixth examples, the reaction chamber is a first reaction chamber and the binding surface is a first binding surface, and wherein the sample container comprises a second reaction chamber having a second binding surface arranged in a symmetric manner with respect to the first axis. In an eighth example of the system, optionally including one or more or each of the first through seventh examples, the first binding surface is symmetric to the second binding surface with respect to a second axis, orthogonal to the first axis, the second axis positioned intermediate the first reaction chamber and the second reaction chamber.

In another representation, a method for a magnetic sensor system includes receiving a sample to be tested in a sample container of the magnetic sensor system, the sample container including a reaction chamber having a binding surface including a first region comprising a first capture element specific to a first analyte and a second region comprising a second capture element specific to a second analyte; activating a magnetic element of the magnetic sensor system positioned below the sample container to pull a plurality of magnetic particles mixed with the sample to the binding surface of the sample container along a magnetic axis, the magnetic axis forming a line of symmetry for the binding surface; and measuring a concentration of the first analyte and the second analyte in the sample, where one or both of the activation of the magnetic element and the measuring are performed while the magnetic sensor system is in motion and/or positioned at a non-flat orientation with respect to gravity. In a first example of the method, the magnetic sensor system is positioned in a vehicle and the magnetic element is activated and/or the measuring is performed while the vehicle is in motion and/or while the vehicle is positioned on an incline or a decline. In a second example of the method, optionally including the first example, measuring the concentration of the first analyte and the second analyte comprises directing light onto the binding surface and detecting light reflected from the binding surface.

References to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to a single one or multiple ones. Additionally, the words “herein,” “above,” “below” and words of similar import, when used in this application, refer to this application as a whole and not to any particular portions of this application. When the claims use the word “or” in reference to a list of two or more items, that word covers all of the following interpretations of the word: any of the items in the list, all of the items in the list and any combination of the items in the list, unless expressly limited to one or the other.

1. A sensor system, comprising:
a sample container configured to receive a sample containing an analyte to be tested, the sample container comprising:
a detection surface; and
a plurality of signal generating elements in the sample container,
wherein the detection surface comprises a binding surface, which has been partially functionalized with capture elements that can bind, directly or indirectly, the analyte or the plurality of signal generating elements;
wherein the plurality of signal generating elements has a spatial distribution profile over the detection surface, wherein the spatial distribution profile has a gradient along a first axis, and
wherein the binding surface has an axis of symmetry that is orthogonal to the first axis.

2. The system of claim 1, wherein the sample container has at least one axis of symmetry, which also constitutes the axis of symmetry of the binding surface.

3. The system of claim 1, wherein the axis of symmetry of the binding surface is a first axis of symmetry, wherein the binding surface has a second axis of symmetry orthogonal to the first axis, and wherein the sample container has two axes of symmetry, orthogonal to each other, which also constitute the first and second axes of symmetry of the binding surface.

4. The system of claim 1, further comprising a magnetic field generation component, able to generate a magnetic gradient within the sample container.

5. The system of claim 4, wherein the magnetic gradient within the sample container causes the plurality of signal generating elements to move towards the first axis of symmetry of the binding surface.

6. The system of claim 4, wherein the second axis of symmetry of the binding surface together with the magnetic gradient causes a number of signal generating elements of the plurality of signal generating elements overlapping the binding surface to remain constant in the presence of external forces such as gravity.

7. The system of claim 1, wherein the spatial distribution profile of the plurality of signal generating elements also has a second gradient along a second axis which is orthogonal to said first axis.

8. The system of claim 7, wherein the binding surface is also symmetric along a third axis that is orthogonal to said second axis.

9. The system of claim 1, wherein the gradient in the spatial distribution profile of the plurality of signal generating elements is such that that the spatial distribution profile has a maximum concentration and then decreases in both directions along the first axis.

10. The system of claim 1, wherein a center of mass of the spatial distribution profile of the plurality of signal generating elements is above the binding surface.

11. The system of claim 1, wherein a fraction of the spatial distribution profile which overlaps the binding surface is 50% or more, 30% or more, 10% or more, or 5% or more.

12. The system of claim 1, further comprising a measurement device to measure an amount of bound signal generating elements and compute a concentration of the analyte.

13. The system of claim 12, wherein the concentration of the analyte is computed using a calibration curve to convert the measured amount of bound signal generating elements into a concentration of the analyte.

14. The system of claim 12, wherein the amount of bound signal generating elements are is measured by the measurement device using fTIR.

15. The system of claim 1, wherein the spatial distribution profile of the plurality of signal generating elements only includes signal generating elements within 100 nm of the detection surface.

16. The system of claim 12, wherein an orientation of the sample cartridge is measured and used to mathematically compensate for the measured amount of bound signal generating elements when computing the concentration of the analyte.

17. The system of claim 1, wherein the binding surface includes a first region which has been functionalized with a first type of capture element and a second region which has been functionalized with a second type of capture element, wherein the first type of capture element can bind, directly or indirectly, the analyte, and the second type of capture element can bind, directly or indirectly, a second, different analyte.

18. A method for a sensor system, comprising:
receiving a sample containing an analyte to be tested in a sample container of the sensor system, the sample container including a detection surface and a plurality of signal generating elements in the sample container, wherein the detection surface comprises a binding surface, which has been partially functionalized with capture elements that can bind, directly or indirectly, the analyte or the plurality of signal generating elements;
generating a spatial distribution profile of the plurality of signal generating elements over the detection surface, wherein the spatial distribution profile has a gradient along a first axis, wherein the binding surface has an axis of symmetry that is orthogonal to the first axis; and
measuring an amount of bound signal generating elements bound to the binding surface and computing a concentration of the analyte in the sample based on the amount of bound signal generating elements.
